(19) United States
(12) Patent Application Publication
(10) Pub. No.: US 2024/0074883 A1
(43) Pub. Date: Mar. 7, 2024

(54) A MODIFIED FIXED FLAT WIRE BIFURCATED CATHETER AND ITS APPLICATION IN LOWER EXTREMITY INTERVENTIONS

(71) Applicant: RAM MEDICAL INNOVATIONS, LLC, Springfield, OH (US)

(72) Inventors: Mubin I. SYED, Springfield, OH (US); Azim SHAIKH, Beavercreek, OH (US); Suresh PAI, Los Altos, CA (US); Celso BAGAOISAN, Union City, CA (US)

(73) Assignee: RAM MEDICAL INNOVATIONS, LLC, Springfield, OH (US)

(21) Appl. No.: 18/386,577

(22) Filed: Nov. 2, 2023

Related U.S. Application Data
(63) Continuation of application No. 17/089,723, filed on Nov. 5, 2020, now Pat. No. 11,877,940.
Application No. 17/089,723 is a continuation of application No. 16/183,066, filed on Nov. 7, 2018, now Pat. No. 10,857,014.
(60) Provisional application No. 62/631,904, filed on Feb. 18, 2018.

(51) Int. Cl. A61F 2/954; A61B 17/3205; A61F 2/966; A61M 25/01

(52) U.S. Cl. CPC A61F 2/954; A61B 17/32056; A61F 2/966; A61M 25/0102; A61M 25/0108; A61M 2025/0681

(57) ABSTRACT

A bifurcated catheter and methods of use are disclosed herein. The bifurcated catheter can include a fixed flat wire that is configurable as a stabilization wire. The bifurcated catheter can be configured to improve the initial access and directability by application of a pull force to the stabilization wire, in addition to a push force from the proximal end of the bifurcated catheter. The stabilization wire is anchored once the bifurcated catheter is positioned. The anchored, bifurcated catheter provides stability and pushability to assist the procedural catheter in traversing the tortuous peripheral vasculature.

### CROSS REFERENCE TO RELATED APPLICATIONS

This application is a continuation of U.S. patent application Ser. No. 17/089,723, entitled “A MODIFIED FIXED FLAT WIRE BIFURCATED CATHETER AND ITS APPLICATION IN LOWER EXTREMITY INTERVENTIONS,” and filed Nov. 4, 2020, which is a continuation of U.S. patent application Ser. No. 16/183,066, entitled “A MODIFIED FIXED FLAT WIRE BIFURCATED CATHETER AND ITS APPLICATION IN LOWER EXTREMITY INTERVENTIONS,” and filed on Nov. 7, 2018, which claims priority under 35 U.S.C. § 119 to U.S. Provisional Application No. 62/631,904, entitled “MODIFIED FIXED FLAT WIRE BIFURCATED CATHETER AND ITS APPLICATION IN AORTO BIFEMORAL BYPASS,” and filed on Feb. 18, 2018, the contents of each of which are hereby incorporated by reference in their entirety.

### TECHNICAL FIELD

The present disclosure relates to improved methods and apparatuses for traversing a tortuous vasculature. Specifically, the present disclosure relates to providing support to procedural catheters during lower extremity intervention procedures to traverse the procedural site through the tortuous access vessels.

### BACKGROUND

Technology associated with interventional procedures is ever developing, particularly in the areas of stenting and balloon angioplasty of Interventional procedures are typically challenging, as accessing various regions of the artery can be dependent on the anatomical disposition of the access location. Specifically, accessing regions of a tortuous peripheral arteries and performing interventional procedures in a hostile anatomy can be very difficult. Furthermore, the subsequent removal of blockages and placement of a stent delivery system into aorto femoral arteries becomes more difficult, or in some instances impossible. The interventional procedure may also be difficult for the popliteal and tibial arteries. The stenting procedure is meant to re-establish a more normalized blood flow through these tortuous arteries by opening up regions constricted by plaque or embolic deposits, which inhibit blood flow.

Although the stent delivery systems are designed to accommodate very acute bends, they are reliant upon guide catheters, guide wires and/or embolic protection devices during deployment. When long delivery systems in tortuous arteries the pushability of catheters and guide wires become critical. As a result, the rigid or stiff catheters and guide wires are needed to manipulate the tortuous entry. With these type of rigid devices, injuries to the tortuous arteries and access vessels often occur during the insertion, manipulation and stabilization of the stent delivery mechanism. Injuries to the tortuous arteries and access vessels often occur during removal of the guide wires, secondary equipment and wires as well. Specifically, the injuries can be caused by puncturing or cutting into the arterial walls resulting in dissections and trauma to the vessels involved. These traumas can be dangerous to the patient as they can ultimately affect blood flow by leakage at the dissections. In some instances, the traumas can create accumulation of thrombus, which is an organization of white blood cells. Dissections and the accumulation of thrombus can require additional procedures to repair and heal the damaged artery walls.

In view of the foregoing, there exists a need to provide a simplified procedure that reduces the injuries caused to the arterial walls during lower extremity interventions. Furthermore, there exists a need to for a usable sheath and catheter stabilization system than enable the use of softer catheters and less stiff guide wires for the treatment of lower extremities.

### BRIEF DESCRIPTION OF THE DRAWINGS

In order to describe the manner in which the above-recited disclosure and its advantages and features can be obtained, a more particular description of the principles described above will be rendered by reference to specific examples illustrated in the appended drawings. These drawings depict only example aspects of the disclosure, and are therefore not to be considered as limiting of its scope. These principles are described and explained with additional specificity and detail through the use of the following drawings.

FIG. 1 illustrates tortuous arteries of the lower extremities, in accordance with an embodiment of the disclosure.

FIG. 2 illustrates a tortuous anatomical pathway from the percutaneous access within the common femoral artery to a potential procedure location on the ipsilateral side, in accordance with an embodiment of the disclosure.

FIG. 3 illustrates a fixed flat-wire bifurcated catheter, in accordance with an embodiment of the disclosure.

FIG. 4 illustrates the fixed flat-wire catheter in the commission of an interventional process, in accordance with one embodiment of the disclosure.

FIG. 5 illustrates a process for introducing a snare catheter and extending a snare wire to an aortic bifurcation, in accordance with an embodiment of the disclosure.

FIG. 6 illustrates a process for inserting a fixed stabilization wire into a main sheath and capturing a stabilization wire extension, in accordance with an embodiment of the disclosure.

FIG. 7 illustrates a process for advancing the bifurcated catheter into the ipsilateral iliac artery, in accordance with one embodiment of the disclosure.

FIG. 8 illustrates a process for advancing the bifurcated catheter into the ipsilateral femoral artery with the stabilization wire externalized and anchored, in accordance with one embodiment of the disclosure.

FIG. 9 illustrates a process for providing stability, tension and pushability of the bifurcated catheter, in accordance with an embodiment of the disclosure.

### DETAILED DESCRIPTION OF ILLUSTRATED EMBODIMENTS

The present invention is described with reference to the attached figures, where like reference numerals are used throughout the figures to designate similar or equivalent elements. The figures are not drawn to scale, and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present disclosure.

The present disclosure provides a system and method for providing access to tortuous arteries to perform lower extremity interventions. The present disclosure is directed towards employing a flat fixed wire bifurcated catheter. The flat fixed wire bifurcated catheter includes a flat wire fixed along the length of the bifurcated catheter from its proximal end to the bifurcation. The flat wire can emerge from a side hole at the bifurcation end of the bifurcated catheter to extend for an additional length beyond the bifurcation. The distal end of the bifurcated catheter can be configured to have a dual lumen (e.g., a large procedural lumen and a small stabilization lumen) from which the flat wire emerges. Alternatively, the distal end of the bifurcated catheter can be configured to have a procedural lumen and a side hole for the stabilization wire.

A small sheath can be provided to enable percutaneous access for the distal end of the support wire. A push and pull force can be applied to the bifurcated catheter to enable the bifurcated catheter to traverse the tortuous vessels to the site of the bifemoral bypass location. This process allows the bifurcated catheter to be positioned in either the common femoral artery or the proximal superficial femoral artery. Furthermore, the stabilization wire can be locked at the accesses to apply tension and stabilization to the procedural lumen.

It should be understood by one of ordinary skill in the art, that the disclosed apparatus can be implemented in any type of lower extremity peripheral arterial intervention. In addition, the disclosed apparatus can be implemented within the internal iliac artery vascular distribution (e.g., uterine artery embolization, prostate artery embolization, proximal internal iliac artery embolization prior to endovascular common iliac artery aneurysm repair, etc.). The disclosed system and method can reduce injury to the vessels within the arteries, reduce trauma caused during the intervention procedure, and improve the procedural success rate. The disclosed system and method can also improve navigation speed through difficult anatomy and enhance stability.

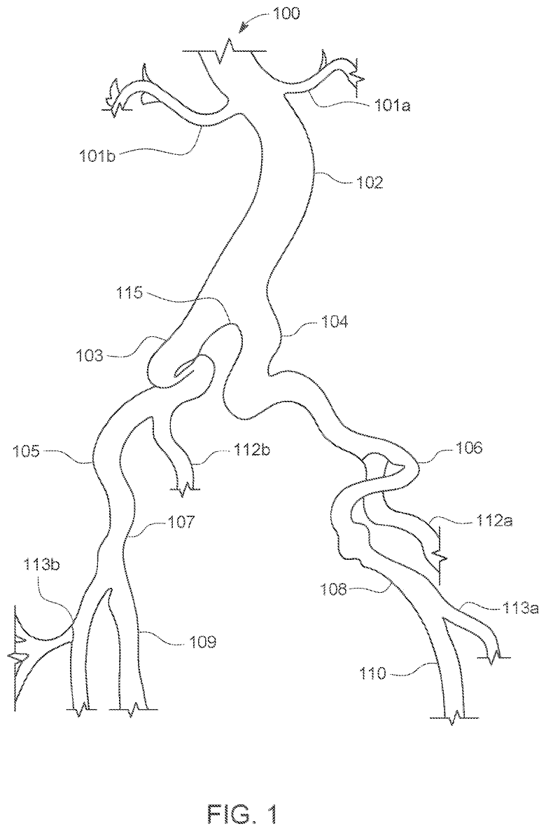

FIG. 1 illustrates an exemplary tortuous vessel artery 100, in accordance with an embodiment of the disclosure. The tortuous vessel artery 100 can include an abdominal aortic bifurcation with tortuous branch arteries. The tortuous branch arteries can include a right renal artery 101b and a left renal artery 101a extending from an abdominal aorta 102. The abdominal aorta 102 can be parted at an aortic bifurcation 115, and connected to arteries of the lower limbs. The arteries of the lower limbs can include a right common iliac 103 and a left common iliac 104. The left common iliac 104 can be split into a left external iliac 106 and a left internal iliac 112a. The left external iliac 106 can be connected to a left common femoral 108, and further split into a left deep femoral 113a, and a left superficial femoral 110. The

The right common iliac 103 can be split into a right external iliac 105 and a right internal iliac 112b. The right external iliac 105 can be connected to a right common femoral 107, which splits into a right deep femoral 113b and a right superficial femoral 109. FIG. 1 illustrates the tortuous nature of the peripheral arteries.

When performing interventions within the tortuous vessel artery 100, it is common to encounter difficulties associated with pushability and torque. As the catheters and wires are guided over a highly angulated aortic bifurcation 115 or through the extremely tortuous common iliac arteries 103 and 104, it can be extremely difficult to apply torque. Furthermore, these arteries can contain calcific plaques or other obstructions which can add anatomic and technical challenges with traversing the tortuous vessel artery 100.

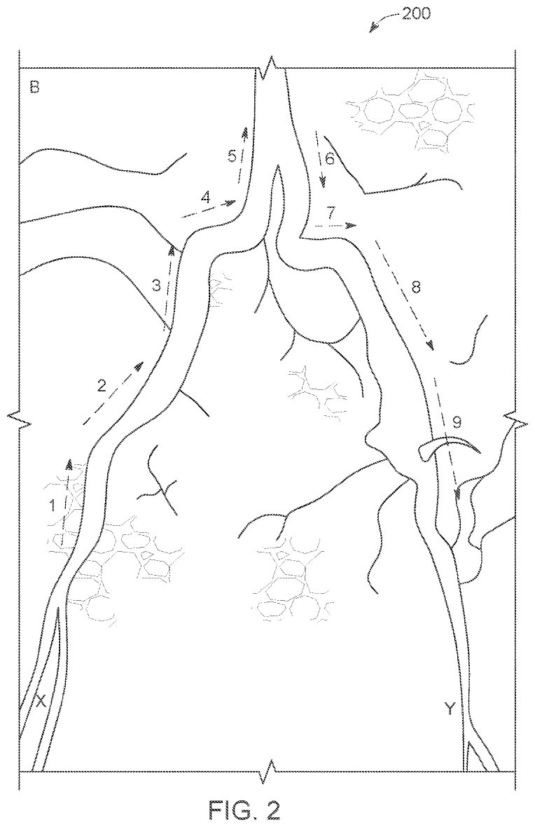

FIG. 2 illustrates a tortuous anatomical pathway 200 from the percutaneous access within the common femoral artery to a potential procedure location on the ipsilateral side, in accordance with an embodiment of the disclosure. In some embodiments, interventional devices such as wires and catheters are pushed from the contralateral access at point ‘X’ to the treatment site ‘Y’. The devices would need to travel through the general pathways 1 through 9. Due to the multi directional twists and turns along the pathways 1 through 9, the devices can suffer from a significant loss of performance such as torque and pushability. While FIG. 2 illustrates the tortuous anatomical pathway 200 in a two-dimensional format, the tortuosity of the anatomical pathway 200 is often significantly more severe, as illustrated in FIG. 1.

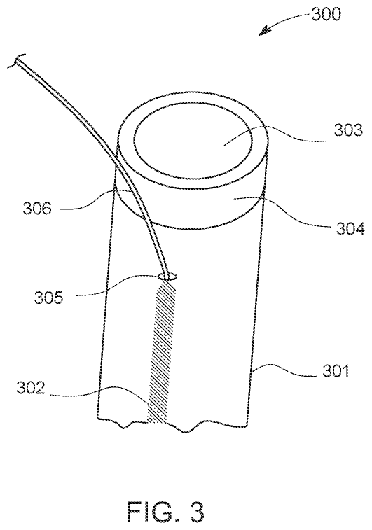

FIG. 3 illustrates a bifurcated catheter 300, in accordance with an embodiment of the disclosure. The bifurcated catheter 300 can include a bifurcated sheath 301 and a fixed flat-wire 302. The fixed flat wire 302 can be integrated within the bifurcated catheter 300. This is discussed in greater detail below.

The bifurcated catheter can have a proximal end (not shown) and a distal end. The distal end can include a side hole 305 for a stabilization wire. The fixed flat wire 302 can be extended from the proximal end (not shown) of the bifurcated catheter 300 to the side hole 305. The bifurcated catheter 300 can also include a bifurcated sheath 301 that spans almost the entire length of the bifurcated catheter 300. In some embodiments, the bifurcation sheath 301 is approximately 2 centimeters from the distal end of the bifurcated catheter 300. For example, in some embodiments the bifurcated catheter 300 can include a radio opaque band 304 at its distal end that limits the length of the bifurcated sheath 301.

The radio opaque band 304 can be implemented to track the distal end of the bifurcated catheter 300 as it is advanced through the arteries of FIG. 1. The bifurcated catheter 300 can also have a procedural lumen 303. In some embodiments, the procedural lumen 303 can extend from its proximal end to its distal end. The bifurcated catheter 300 can also have a stabilization wire 306. The stabilization wire 306 can be configured to protrude from the bifurcated catheter 300 at the side hole 305. In some embodiments, the stabilization wire 306 can extend up to 10 cm beyond the end of the bifurcated catheter 300.

Furthermore, the stabilization wire 306 can be a flat wire or a round wire. For example, the stabilization wire 306 can be made up of a solid or hollow member with a cross-section that is round, flat, rectangular, or a combination thereof. The stabilization wire 306 can be fabricated using commonly known materials in the art including, for example, stainless steel, nickel titanium, composites, metal reinforced polymer, polymer, a combination thereof, or the like.

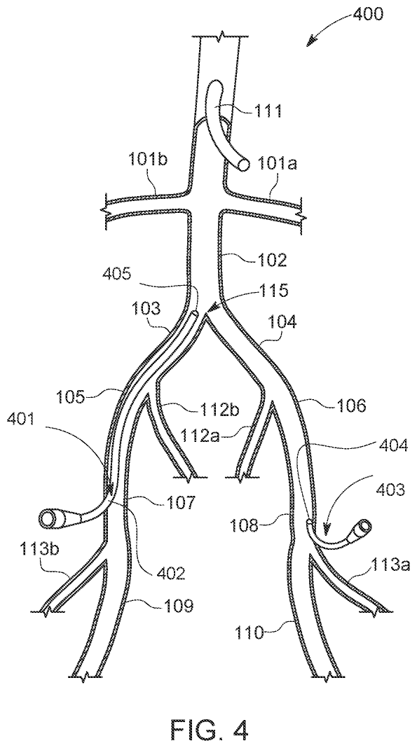

FIGS. 4 to 8 illustrate an exemplary process for lower extremity intervention implementing the bifurcated catheter 300 of FIG. 3. The lower extremity intervention can include, for example, an Aorto Bifemoral Bypass or a tortuous aortoiliac artery treatment. Furthermore, FIGS. 4 to 8 illustrate the process of providing end-to-end stability to any additional procedural catheter and instruments introduced through the procedural lumen 303 of the bifurcated catheter 300.

FIG. 4 illustrates a diagram 400 where a percutaneous contralateral femoral access 402 is introduced into the right common femoral artery 107. The percutaneous contralateral femoral access 402 can be implemented to introduce a main access sheath 401 into the right common femoral artery 107. The main access sheath 401 can be configured as a 7Fr. vascular sheath. The main access sheath 401 can be advanced through the right external 105 and right common iliac 103 to the aortic bifurcation 115. The main access sheath 401 can be tracked using the radio opaque band 304 as the main access sheath 401 is advanced to the aortic bifurcation 115. FIG. 4 also illustrates the introduction of a percutaneous ipsilateral femoral access 404 into the left common femoral artery 108. The percutaneous ipsilateral femoral access 404 can be introduced for a snare access sheath 403 of a 4Fr. internal lumen.

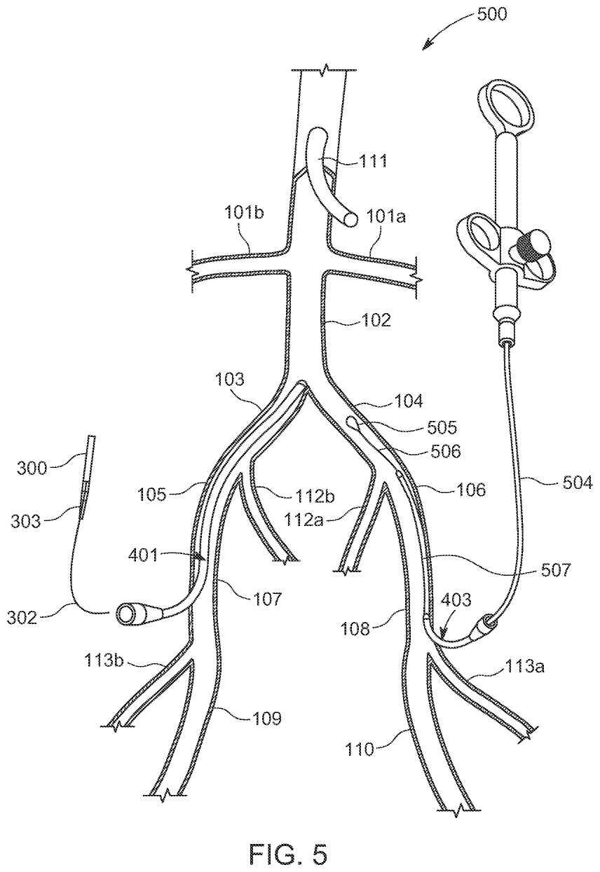

FIG. 5 illustrates a process for introducing a snare catheter and extending a snare wire to an aortic bifurcation, in accordance with an embodiment of the disclosure. Once the access sheaths 401 and 403 are in place, a 4Fr snare sheath 504 and snare wire 506 can be introduced through the retrograde snare access sheath 403. In some embodiments, the snare wire 506 can include a snare 505 at its distal end extending to the aortic bifurcation 115. The snare 505 can be 20 to 30 mm (or smaller) in length. In some embodiments, the fixed flat wire bifurcated catheter 300 can include a dilator 503 in the main sheath. The fixed flat wire bifurcated catheter 300 and the stabilization wire 306 can be introduced through the main access sheath 401. The main access sheath 401 can include a distal end, labeled as the tip of the sheath aligned to the aortic bifurcation. FIG. 5 further illustrates the fixed flat wire bifurcated catheter 300 and the stabilization wire 306 being pushed through the distal end of the main sheath 401. The stabilization wire 306 can be extended out of the distal end of the main sheath 401 to be captured at the distal end of the snare 505.

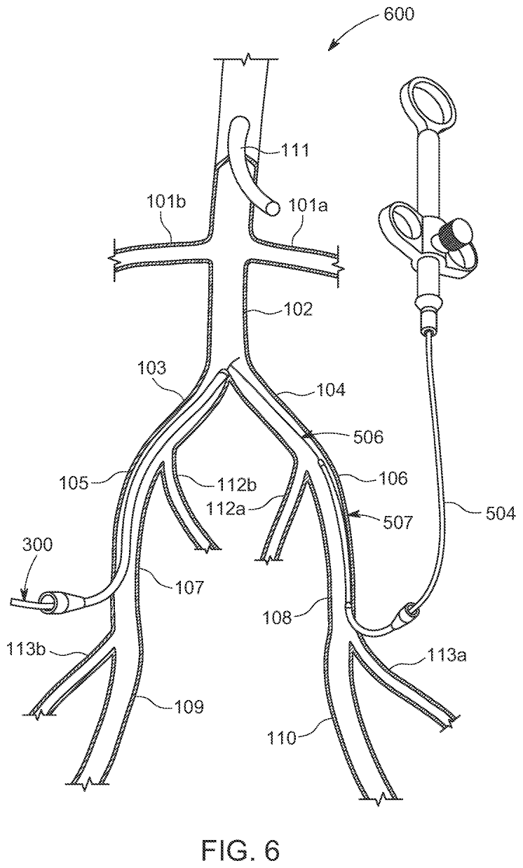

FIG. 6 illustrates a process for inserting a fixed stabilization wire into a main sheath and capturing a stabilization wire extension, in accordance with an embodiment of the disclosure. The stabilization wire 306 can be tightened to a snare knot 601. A pull force 703 can be applied to the distal end of the bifurcated catheter 300. The pull force 703 can be derived from the snare catheter 504 and the snare wire 506, which has snared the stabilization wire 306. A push force 701 can be applied on the proximal end of the bifurcated catheter 300. The push force 701 and the pull force 703 can be applied simultaneously. The push force 701 and the pull force 703 guides the distal end of the bifurcated catheter 300 with the dilator tip 503 over the aortic bifurcation and down the ipsilateral left iliac arteries 104 and 105.

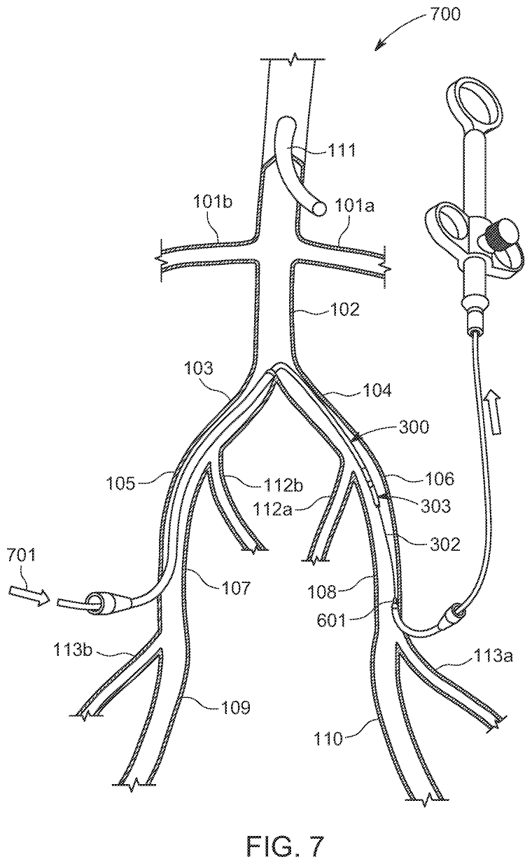

FIG. 7 illustrates a process for advancing the bifurcated catheter into the ipsilateral iliac artery, in accordance with one embodiment of the disclosure. Once guided over the aortic bifurcation and down the ipsilateral left iliac arteries 104 and 105, the bifurcated catheter 300 can be guided to the left common femoral artery 108. The snare wire 506, encompassing the snared stabilization wire 306, can be pulled out of the ipsilateral snare access sheath 403 and anchored.

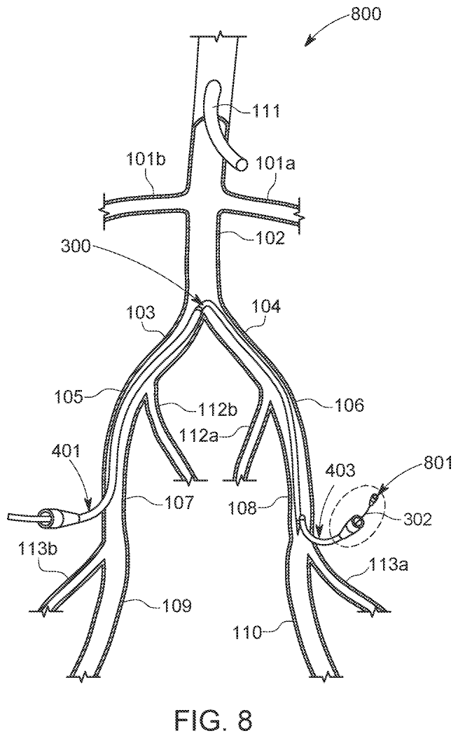

FIG. 8 illustrates a process for advancing the bifurcated catheter into the ipsilateral femoral artery with the stabilization wire externalized and anchored, in accordance with one embodiment of the disclosure. The snare wire 506 can be anchored with the snared stabilization wire 306 locking the snare wire external to the snare access sheath. The snare wire can be locked to the snare access sheath using a wire lock 801. By locking the stabilization wire 306 outside the snare access 403 and providing a pull force on the distal end of the bifurcated catheter, a tension can be applied to the main procedural lumen 303. This tension can provide stabilization to the main procedural lumen 303. Any procedural catheters and instruments within the main procedural lumen 303 can also be stabilized. Anchoring and locking the stabilization wire can cause bifurcation of the bifurcated catheter at the snare access sheath. This bifurcation can provide an anchor point for improved pushability to the procedural catheters.

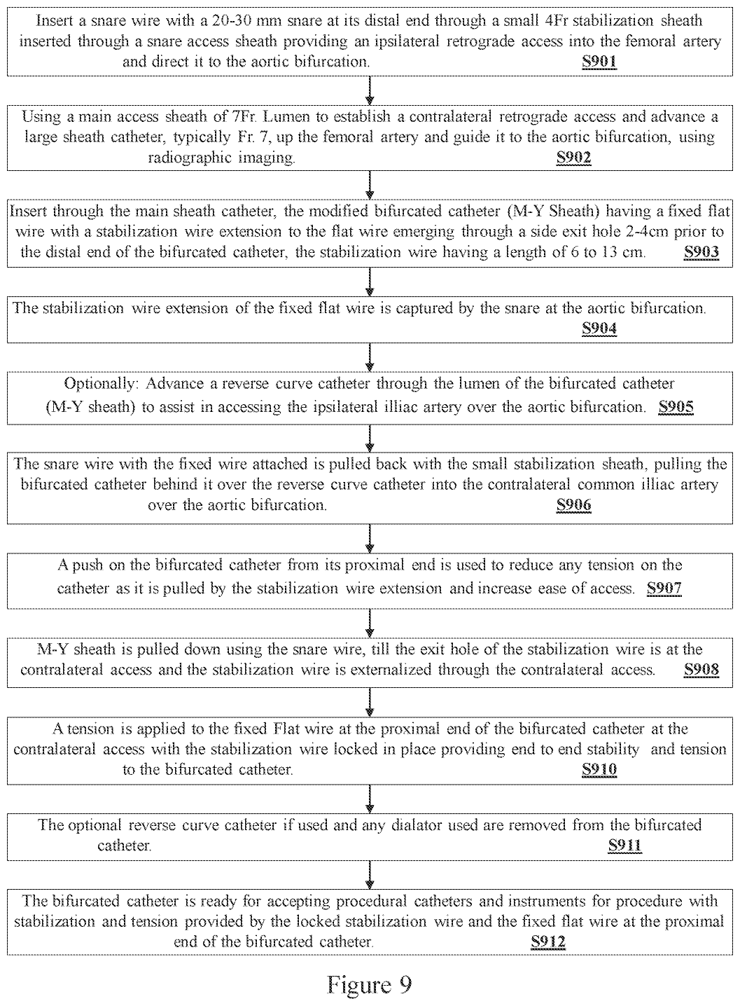

FIG. 9 illustrates a process 900 for providing stability, tension and pushability of the bifurcated catheter of FIG. 3, in accordance with an embodiment of the disclosure.

At step 901, a small 4 Fr lumen snare access sheath is inserted into the left common femoral artery to provide an ipsilateral retrograde access. A snare catheter is inserted through the snare access sheath with a snare wire. The snare catheter can be 4 Fr or smaller. The snare wire can have a 20-30 mm snare at its distal end. In some embodiments, the snare wire can have a snare less than 20 mm at its distal end. The snare wire can be guided to the aortic bifurcation.

At step 902, a main access sheath can be used to establish a contralateral retrograde access at the right common femoral artery location. The main access sheath can be a 7Fr. Lumen. A large sheath catheter can be advanced up the right femoral artery and the iliac arteries towards the aortic bifurcation. The large sheath catheter can be guided using radiographic imaging. The large sheath catheter can be Fr.7.

At step 903, a modified bifurcated catheter can be inserted into the main sheath and guided to the aortic bifurcation. The modified bifurcated catheter can have a fixed flat wire secured to the bifurcated catheter, from its proximal end to the bifurcation. An extension of the flat wire can emerge through a side exit hole at the bifurcation. The extension can be the stabilization wire. The side exit hole at the bifurcation can be between 2-4 cm prior to the distal end of the bifurcated catheter. In some embodiments, the stabilization wire can have a length of 6 to 13 cm beyond the side exit hole. In alternative embodiments, the stabilization wire can be extended beyond 13 cm from the side exit hole.

At step 904 the stabilization wire, extending from the side exit hole, can be captured by the snare of the snare wire. The snare wire can capture the stabilization wire at the aortic bifurcation. The snare can be tightened to secure the stabilization wire and apply a pull pressure on it.

At step 905 a reverse curve catheter can be inserted through the main lumen of the bifurcated catheter to access the left common iliac artery. The reverse curve catheter can assist in transitioning the bifurcated catheter from the contralateral right common iliac artery to the ipsilateral left common iliac artery over the aortic bifurcation.

At step 906, an external pull force can be applied on the snare wire with the stabilization wire snared. The pull force can be accomplished by implementing a small stabilization sheath. By pulling the small stabilization sheet, the attached fixed flat wire and the main lumen are also pulled over the reverse curve catheter into the contralateral common left iliac artery.

At step 907 a push force is applied on the bifurcated catheter at its proximal end to assist advance the bifurcated catheter past sharp corners and reduce tension on the catheter as it is pulled by the stabilization wire. The combination of the push and pull force enable the bifurcated catheter to easily overcome obstructions as it traverses tortuous curves of the vessels. The combination of push and pull forces also help to reduce the tension on the bifurcated catheter and increase access while reducing the trauma to the vessels.

At step 908 the bifurcated sheath is simultaneously pulled and pushed down the ipsilateral left side vasculature, until the side exit hole of the stabilization wire is at the ipsilateral snare access sheath location. The stabilization wire is externalized by pulling the snare wire out of the access sheath through the ipsilateral access.

At step 909 the externalized stabilization wire is anchored by locking it in place at the ipsilateral access by a wire lock. The modified bifurcated catheter is anchored at the distal end of the bifurcation.

At step 910, a tension is applied to the fixed Flat wire at the proximal end of the bifurcated catheter. The tension is applied at the contralateral access with the stabilization wire. The stabilization wire can be locked in place at the distal end. An end-to-end application of tension can be applied to provide stability to the bifurcated catheter. This stability increases pushability of any procedural catheters within its main procedural lumen.

At step 911 any optional reverse curve catheter can be used. Furthermore, any dilator used to reduce trauma to vessels can be removed from the bifurcated catheter.

At step 912 the bifurcated catheter is configured to accept the procedural catheters and instruments for procedure, through its main lumen. Stabilization and tension can be provided by the locked stabilization wire at the distal end of the bifurcation and the fixed flat wire. The bifurcated catheter is configured for interventional procedures (stents, atherectomy, etc.) within the left peripheral vasculature. The process 900 is terminated after step 912.

The examples provided herein are directed towards specific examples. One of ordinary skill in the art would understand the provided examples are not intended to be exhaustive. There exists other exemplary access and stabilization of a procedural catheter or sheath. As is well understood, the preferred method will vary based on the location of the procedure and the physical condition of the patient.

As is well understood by those familiar with the art, the invention may be embodied in other specific forms without departing from the spirit or essential characteristics thereof. Likewise, the naming and division of the members, features, attributes, and other aspects are not mandatory or significant, and the mechanisms that implement the invention or its features may have different structural construct, names, and divisions. Accordingly, the disclosure of the invention is intended to be illustrative, but not limiting, of the scope of the invention.

The embodiments disclosed herein can be implemented as hardware, firmware, software, or any combination thereof. Moreover, the software is preferably implemented as an application program tangibly embodied on a program storage unit or computer readable medium. The application program may be uploaded to, and executed by, a machine comprising any suitable architecture.

1. A system comprising:
a bifurcated catheter comprising a proximal end and a distal end;
the bifurcated catheter further comprising a side hole near the distal end;
a stabilization wire attached to a wall of the bifurcated catheter, the stabilization wire configured to emerge and extend from the side hole;
a snare access sheath configured to have a snare catheter comprising a snare wire inserted therethrough;
wherein the snare wire is configured to snare a distal end of the stabilization wire using a snare knot;
wherein the stabilization wire is configured to be pulled to guide the bifurcated catheter;
wherein the snared stabilization wire is configured to be pulled and externalized by the snare wire to lock the distal end of the stabilization wire at a first location; and
wherein the bifurcated catheter is configured to be locked at a second location to enable application of tension and provide stabilization to the bifurcated catheter.

2. The system of claim 1, wherein the side hole is at a distance between 1 and 3 cm from the distal end of the bifurcated catheter.

3. The system of claim 1, wherein the stabilization wire comprises two sections, a first section fixed to the wall of the bifurcated catheter and a second section that is outside the side hole of the bifurcated catheter.

4. The stabilization wire of claim 3, wherein the first section of the stabilization wire is configured to have a flat cross section.

5. The stabilization wire of claim 3, wherein the second section of the stabilization wire is configured to have a circular cross section.

6. The stabilization wire of claim 3, wherein the second section of the stabilization wire is configured to extend 8 to 14 cm beyond the side hole.

7. The stabilization wire of claim 3, wherein the second section of the stabilization wire extends beyond the distal end of the bifurcated catheter.

8. The system of claim 1, wherein the bifurcated catheter is configured for access into the vasculature via a percutaneous contralateral access.

9. The system of claim 8, wherein the second location is outside the percutaneous contralateral access to the vasculature.

10. The system of claim 1, wherein the distal end of the bifurcated catheter comprises a radio opaque band to enable guiding the distal end of the bifurcated catheter.

11. The system of claim 8, wherein the snare catheter is configured for an access into the vasculature via an ipsilateral percutaneous access.

12. The system of claim 8, wherein the first location where the snare wire is externalized and locked is outside the ipsilateral percutaneous access to the vasculature.

13. The system of claim 11, wherein the percutaneous contralateral access and ipsilateral percutaneous access are arterial accesses.

14. The system of claim 1, wherein the bifurcated catheter is configured to receive a pull force from the stabilization wire and a push force from the proximal end of the bifurcated catheter to advance the bifurcated catheter through tortuous vessels to a site of a lower extremity intervention procedure.

15. The system of claim 1, wherein the stabilization wire is configured to apply tension and stabilization to the procedural catheter for access to a procedural location.

16. The system of claim 1, wherein the proximal end of the stabilization wire is configured to be affixed to the bifurcated catheter wall from a third location within the bifurcated catheter between the proximal and distal ends of the bifurcated catheter, with the distal end of the stabilization wire configured to extend from the side hole beyond the distal end of the bifurcated catheter.

17. A system for providing tension and stabilization to a procedural catheter, the system comprising:
a bifurcated catheter having a proximal end and a distal end, the bifurcated catheter configured to be inserted via a contralateral arterial access;
the bifurcated catheter having a side hole between 1 to 4 centimeters (cm) from the distal end;
the stabilization wire comprising two sections, a proximal section configured to attach to a wall of the bifurcated catheter, and a distal section configured to exit via the side hole, the stabilization wire configured to extend for 8 to 14 cm beyond the side hole;
a snare access sheath configured to have a snare catheter comprising a snare wire inserted therethrough, the snare access sheath configured for an ipsilateral arterial access;
the snare wire configured to snare a distal end of the stabilization wire and externalize and lock the stabilization wire outside the ipsilateral arterial access;
the proximal end of the bifurcated sheath configured to be locked outside the contralateral arterial access;
wherein the locked bifurcated sheath and the locked stabilization wire together provide access, tension and stabilization to a procedural catheter and instruments used for a lower extremity intervention procedure.

18. The system of claim 17, wherein when the stabilization wire is snared and externalized it is configured to guide the bifurcated catheter through the tortuous vasculature of the lower extremity.

19. The system of claim 17, wherein a push-pull force using the stabilization wire and the bifurcated catheter enable easy access for the procedural catheter and instruments to the site of the lower extremity intervention procedure.

20. The system of claim 17, wherein the stabilization wire locked outside the ipsilateral arterial access is configured to provide stabilization to the bifurcated catheter and the procedural catheter during the lower extremity intervention procedure.
